(19) United States
(12) Patent Application Publication
(10) Pub. No.: US 2017/0181822 A1
(43) Pub. Date: Jun. 29, 2017

(54) MICRO-NEEDLE DEVICE

(71) Applicant: 3M INNOVATIVE PROPERTIES COMPANY, St. Paul, MN (US)

(72) Inventors: Marc Peuker, Schoendorf (DE); Andreas Syrek, Munich (DE); Martin Preininger, Bavaria (DE); Mathias Bertl, Wildsteig (DE); Emir Jelovac, Munchen (DE); Ryan P. Simmers, Fargo, ND (US); Boon Yi Soon, Jurong East (SG)

(21) Appl. No.: 15/124,404

(22) PCT Filed: Mar. 5, 2015
(86) PCT No.: PCT/US15/18899

§ 371 Date: Sep. 8, 2016

Related U.S. Application Data
(60) Provisional application No. 61/950,426, filed on Mar. 10, 2014.

(51) Int. Cl. A61C 19/08; A61M 37/00

(52) U.S. Cl. CPC A61C 19/08; A61M 37/0015; A61M 2037/0023; A61M 2210/0625; A61M 2210/0631; A61M 2202/048; A61M 2037/003; A61M 2037/0046

(57) ABSTRACT

The present disclosure provides for a micro-needle device (100, 200, 300, 400) that includes a micro-needle array (102, 202, 302, 402) and a liquid connection port (104, 204, 304, 404). The micro-needle array includes a base (106, 206, 306, 406), a sidewall (108, 208, 308, 408) and a top (110, 210, 310, 410), where the base includes two or more of an elongate micro-needle (112, 212, 312, 412) having an interior surface (114, 214, 314, 414) defining an opening 8116, 216, 316, 416) through the elongate micro-needle. The base has a first major surface (118, 218, 318, 418) and a second major surface (120, 220, 320, 420) through which the opening of the elongate micro-needle passes to provide a passage across the base. The top has an interior surface (122, 222, 322, 422) and the sidewall has an interior surface (126, 226, 326, 426), where the interior surface of the side wall, the interior surface of the top and the first major surface of the base define a volume (130, 230, 330, 430). The liquid connection port has a fluid connection with the volume of the micro-needle array such that dental local anesthetic fed through the connection port can exit through the opening of the elongate micro-needle.

### FIELD OF THE DISCLOSURE

The disclosure relates to a medical device and in particular to a medical device having micro-needles.

### BACKGROUND ART

Needles sometimes need to be used for injections during medical procedures. The sight, thought and/or feeling of a needle can cause fear in the patient. This fear, or phobia, of needles is known as needle phobia.

Depending upon the degree of needle phobia, a patient can display a wide variety of symptoms. For example, a patient with needle phobia can have anxiety, a panic attack, an elevated blood pressure and/or an elevated heart rate knowing that a needle may or will be used in their medical procedure. In extreme cases the patient can faint due to a vasovagal reflex reaction. This leads to an unsafe situation for both the patient and the medical personnel. Other reactions of patients with needle phobia can include avoiding medical treatment if they know or believe a needle will be used. In extreme cases, some patients will avoid all medical care. This fear of needles can also be associated with the sight of a syringe.

In dentistry, a syringe fitted with a needle is often times used to deliver an anesthetic to the patient. The needle and syringe are inserted at least partially into the patient's mouth, where the needle is inserted into the gingiva and/or other tissues (e.g., oral mucosa) in order to deliver a local anesthetic. Using a local anesthetic can help to decrease intraoperative and postoperative pain, decrease the amount of general anesthetics used in the operating room, increase the patient cooperation during the procedure. Often times the injection is more painful and traumatic than the actual procedure.

Therefore, there is a need in the art for a suitable device for injecting a local anesthetic that does not use a traditional needle and syringe configuration, which configurations are well known to cause issues with many patients.

### SUMMARY OF THE DISCLOSURE

The present disclosure provides a device for delivering a dental local anesthetic that does not use a traditional needle and syringe configuration. For example, the micro-needle device of the present disclosure does not include a plunger.

The present disclosure provides a micro-needle device for delivering a dental local anesthetic that includes a micro-needle array having a base, a sidewall and a top. The base includes two or more of an elongate micro-needle, the elongate micro-needle having an interior surface defining an opening through the elongate micro-needle and the base having a first major surface and a second major surface through which the opening of the elongate micro-needle passes to provide a passage across the base. The top has an interior surface and the sidewall has an interior surface, where the interior surface of the side wall, the interior surface of the top and the first major surface of the base define a volume.

The liquid connection port provides a fluid connection with the volume of the micro-needle array such that dental local anesthetic fed through the connection port can exit through the opening of the elongate micro-needle. The liquid connection port extends from the sidewall of the micro-needle array. The micro-needle device can further include a catheter that extends from the liquid connection port to a first end, where the catheter provides a fluid connection from the first end to the volume of the micro-needle array. A syringe can be releasably coupled to the first end of the catheter to provide the fluid connection with the volume of the micro-needle array.

The micro-needle array can further include a spring that connects the micro-needle array and a button positioned over the top of the micro-needle array, where the spring compresses under pressure applied through the button and against the micro-needle array when the micro-needle device is positioned in a mouth of a patient. The top can include an exterior surface opposite the second major surface of the base, the exterior surface of the top having a protrusion that extends towards the button positioned over the top of the micro-needle array. The micro-needle array can further include a finger ring that extends from the spring, where the finger ring holds a finger against the button. The finger ring can have a first arm and a second arm that form a hoop of the finger ring.

The button can have a surface defining an opening through the button, where the protrusion passes at least partially through the opening in the button when the spring is compressed under pressure applied through the button and against the micro-needle array when the micro-needle device is positioned in a mouth of a patient. The top of the micro-needle device includes an exterior surface opposite the second major surface of the base, the exterior surface of the top having a pressure sensitive adhesive for retaining the micro-needle device on a user's finger.

### BRIEF DESCRIPTION OF THE FIGURES

The Figures may not be to scale.

FIG. 1A is a perspective view of a micro-needle device according to an embodiment of the present disclosure.

FIG. 1B is a cross sectional view of the micro-needle device taken along lines 1B in FIG. 1A.

FIG. 1C is a plane view of the micro-needle device of FIG. 1A, a catheter and a syringe according to an embodiment of the present disclosure.

FIG. 2 is a perspective view of a micro-needle device according to an embodiment of the present disclosure.

FIG. 3 is a perspective view of a micro-needle device according to an embodiment of the present disclosure.

FIG. 4 is a cross-sectional view of a micro-needle device according to an embodiment of the present disclosure.

### DETAILED DESCRIPTION OF THE DISCLOSURE

The micro-needle device of the present disclosure may be used to inject a local anesthetic without using a traditional needle and syringe configuration. As disclosed herein, the micro-needle device has a non-medical device appearance, but yet enables the delivery of a dental local anesthetic to the oral tissues of a patient. The micro-needle device of the present disclosure provides a micro-needle array having a low profile that allows for discrete handling and insertion into the patients mouth. As such, a patient having needle phobia may be less likely to react negatively and/or be more willing to undergo a dental procedure because the traditional needle and syringe configuration will not be used.

The micro-needle device also includes a liquid connection port associated with the micro-needle array. The liquid connection port allows for a liquid (e.g., dental local anesthetic) to be injected through the micro-needle array. It is also possible to use a catheter with the liquid connection port, where a free end of the catheter can include a fluid fitting to allow a syringe to be releasably attached to the micro-needle device. Given an appropriated length of the catheter the syringe can be located out of sight of the patient. This option of locating the syringe out of sight of the patient along with the low profile nature of the micro-needle device of the present disclosure will potentially help those patients who have needle phobia.

As used herein, “a,” “an,” “the,” “at least one,” and “one or more” are used interchangeably. The term “and/or” means one, one or more, or all of the listed items. The recitations of numerical ranges by endpoints include all numbers subsumed within that range (e.g., 1 to 5 includes 1, 1.5, 2, 2.75, 3, 3.80, 4, 5, etc.).

As recited herein, all numbers can be considered to be modified by the term “about.”

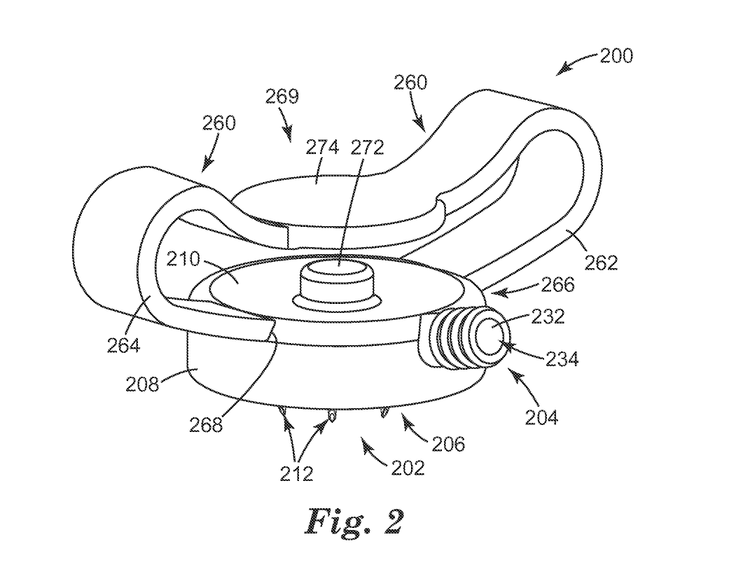

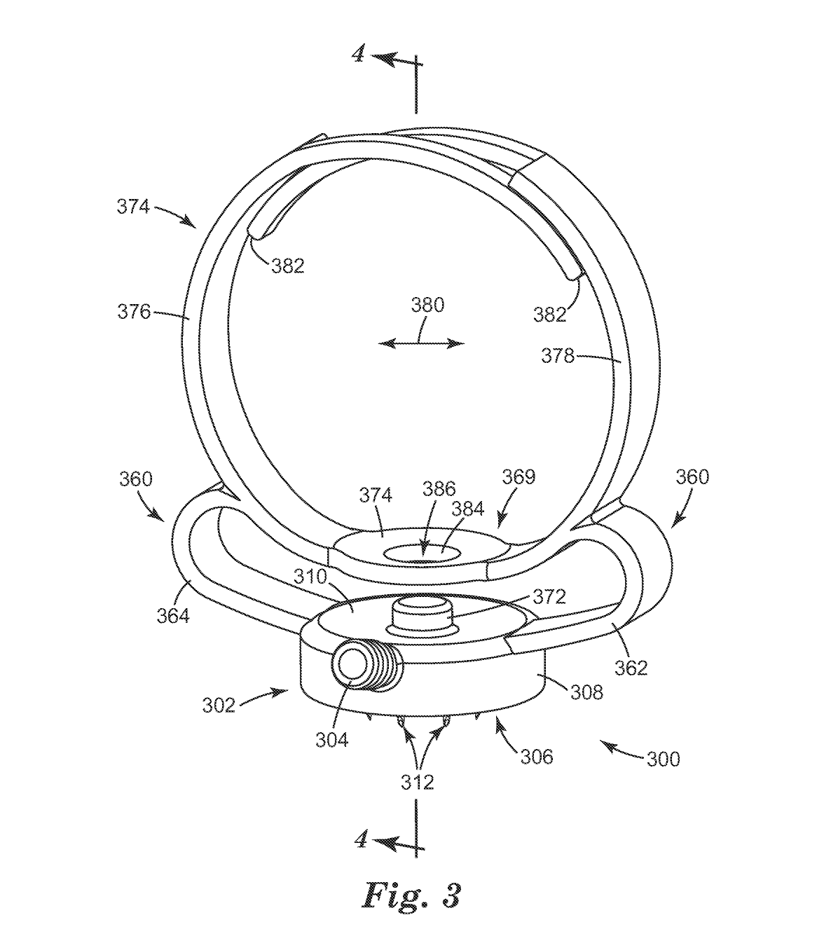

The figures herein follow a numbering convention in which the first digit or digits correspond to the drawing figure number and the remaining digits identify an element or component in the drawing. Similar elements or components between different figures may be identified by the use of similar digits. For example, 214 may reference element “14” in FIG. 2, and a similar element may be referenced as 314 in FIG. 3. Elements shown in the various figures herein can be added, exchanged, and/or eliminated so as to provide a number of additional examples of the present disclosure. In addition, the proportion and the relative scale of the elements provided in the figures are intended to illustrate the examples of the present disclosure, and should not be taken in a limiting sense.

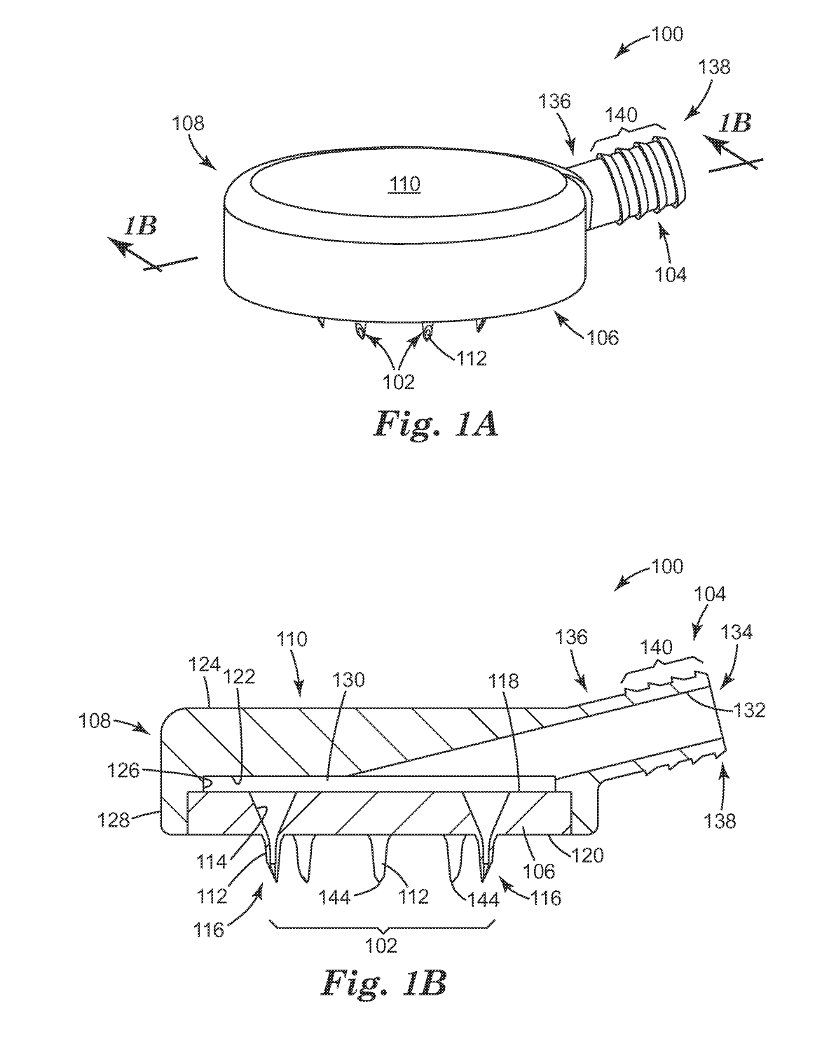

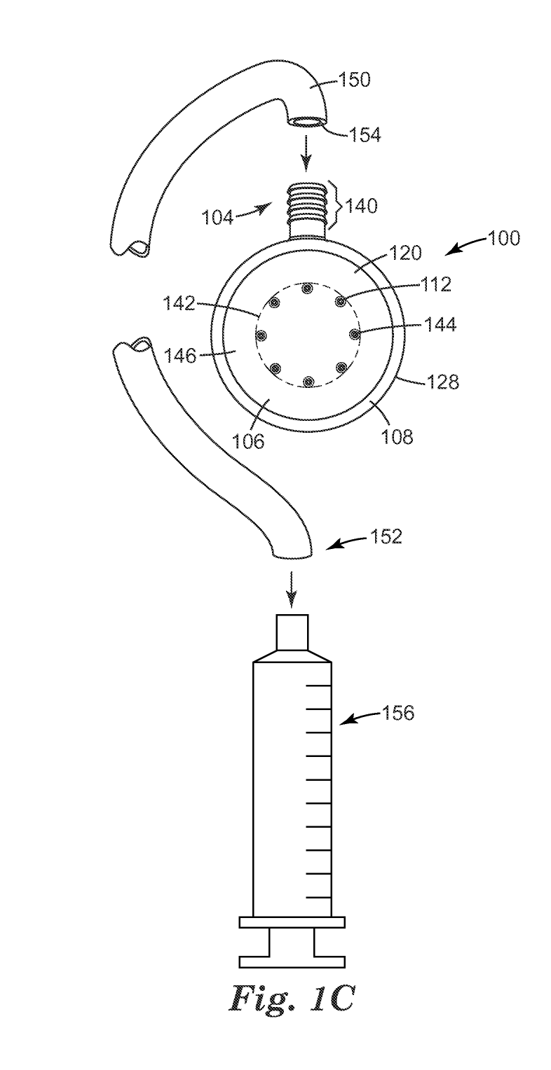

Referring now to FIGS. 1A-1C, there is shown an embodiment of a micro-needle device 100 for delivering a dental local anesthetic. The micro-needle device 100 includes a micro-needle array 102 and a liquid connection port 104. The micro-needle array 102 includes a base 106, a sidewall 108 and a top 110. The base 106 includes two or more of an elongate micro-needle 112. The elongate micro-needle 112 has an interior surface 114 defining an opening 116 through the elongate micro-needle 112. The base 106 has a first major surface 118 and a second major surface 120 through which the opening 116 of the elongate micro-needle 112 passes to provide a passage across the base 106. The top 110 has an interior surface 122 and an exterior surface 124.

The sidewall 108 has an interior surface 126 and an exterior surface 128. The interior surface 126 of the sidewall 108, the interior surface 122 of the top 110 and the first major surface 118 of the base 106 define a volume 130.

The liquid connection port 104 includes a lumenal surface 132 defining a lumen 134 that is in fluid connection with the volume 130 of the micro-needle array 102. This allows dental local anesthetic fed through the liquid connection port 104 to pass through the lumen 134, into the volume 130 and exit through the opening 116 of the elongate micro-needle 112.

A problem with traditional needle structures is that the connector of the needle (e.g., a Luer connector) is aligned with the needle along the direction through which the force is applied to insert the needle into the patient. In order to apply this force and inject the substance into the patient a syringe is joined to the needle. Once the syringe is joined to the needle the structure is so long that the patient could not help noticing it. The sight of this very long structure with its needle can be of great concern for those people with needle phobia.

In contrast to traditional needle and syringe structure, the micro-needle array 102 of the present disclosure has a disk-shape. As illustrated, the exterior surface 128 of the sidewall 108, the exterior surface 124 of the top 110 and the second major surface 120 of the base 106 give the micro-needle array 102 this disk-shape. The disk-shape provides a relatively large surface on which the doctor can both hold the micro-needle device 100 (via the exterior surface 128 of the sidewall 108) and apply force (via the exterior surface 128 of the top 110) to insert the micro-needles 112 in the oral tissue of the patient. One advantage of this disk-shape is that the doctor can discretely hold the micro-needle array 102 in a position that also allows them to use the micro-needle device 100.

Another advantageous feature of the micro-needle device 100 is that the liquid connection port 104 does not extend in the direction along which the force is applied to insert the micro-needles 112 into the tissue of the patient. In other words, the liquid connection port 104 is outside the exterior surface 124 of the top 110 (e.g., the pressure area of the micro-needle device 100). For example, as illustrated in FIGS. 1A and 1B the liquid connection port 104 extends not from the top 110 of the micro-needle array 102, but from the sidewall 108 of the micro-needle array 102. This allows for almost the entire exterior surface 124 of the top 110 to be available to the doctor. An additional advantage is also that the doctor can apply force via the exterior surface 124 of the top 110 to insert the micro-needles 112 into the tissue of the patient without having to simultaneously dispense the substance, as is the case with other micro-needle devices.

It is appreciated that other flat thin shapes may also be used instead of a disk-shape for the micro-needle array 102. For example, the micro-needle array 102 may have, as viewed perpendicular to the exterior surface 124 of the top 110, an oval shape, an elliptical shape, a polygon shape such as a rectangular shape or a square shape. The exact shape of the micro-needle array 102 can be determined based on the desired use and location of the use for the micro-needle device 100.

The exterior surface 124 can also have a variety of shapes. For example, the exterior surface 124 can have a planar shape. Alternatively, the exterior surface 124 can have a concave shape. The concave shape can help to better center a finger (e.g., an index finger) that is used to press on the micro-needle array 102. Other geometrical shapes can be used for the exterior surface 124 that would help as a finger guide.

As illustrated, the liquid connection port 104 extends away from the micro-needle array 102 in a manner that allows the liquid connection port 104 to connect to a fluid source (e.g., a catheter and syringe as discussed herein) without having the components of the fluid source extend, relative the top 110, beyond the second major surface 120 of the base 106. So, for example, the liquid connection port 104 can include an elbow 136 that helps to project a distal end 138 of the liquid connection port 104 away from the base 106. As illustrated, the liquid connection port 104 near the distal end 138 can include a fluid fitting 140 to receive and retain a catheter (seen in FIG. 1C). FIGS. 1A-1C illustrate the fluid fitting 140 as a series of circular barbs. It is also appreciated the outer diameter of the liquid connection port 104 can taper to present a distal end 138 having a diameter that is smaller than a portion of the port 104 that meets with the sidewall 108. Other fluid fittings 140 are possible, such as a female part or male part of a Luer Taper connector (either a “Luer-Lok” or “Luer-Slip” configuration).

The base 106 and the top 110 of the micro-needle array 102, in the disk-shape can, have a diameter of 4 millimeters (mm) to 15 mm, where the sidewall 108 can have a height of 0.5 mm to 8 mm. Preferably, the base 106 and the top 110 of the micro-needle array 102, in the disk-shape can, have a diameter of 5 mm to 10 mm, where the sidewall 108 can have a height of 1 mm to 6 mm. Most preferably, the base 106 and the top 110 of the micro-needle array 102, in the disk-shape can, have a diameter of 6 mm to 8 mm, where the sidewall 108 can have a height of 2 mm to 4 mm.

The base 106 of the micro-needle array 102 has 6 to 18 micro-needles 112. The second major surface 120 of the base 106 includes an outer boundary 142 (shown with a broken line in FIG. 1C) that along with the exterior surface 128 of the sidewall 108 define an infiltration area 146 (the area that extends from the outer boundary 142 to the exterior surface 128 of the sidewall 108). As discussed herein, the exterior surface 124 of the top 110 is opposite the second major surface 120 of the base 106. The exterior surface 124 of the top 110 provides a continuous surface which can receive pressure from a finger and also where the exterior surface 124 opposite of the second major surface 120 and the infiltration area 146 overlap each other by at least 75%. So, for example, when the exterior surface 124 of the top 110 has the same size and shape of the second major surface 120 of the base 106 and the sidewall 108 is perpendicular to both the exterior surface 124 and the second major surface 120 there is an overlap of 100%. If one of either the exterior surface 124 of the top 110 or the second major surface 120 of the base 106 has a different size and/or shape then the overlap of these areas should be at least 75%.

The micro-needles 112 of the micro-needle array 102 can have variety of patterns. For example, the micro-needles 112 can be uniformly arranged in a circular pattern to help define the infiltration area 146, as illustrated in FIG. 1C. In this embodiment, the circular pattern is centric relative the geometric center of the second major surface 120 of the base 106. If desired, the pattern of the micro-needles 112 can be either centric or eccentric relative the geometric center of the second major surface 120 of the base 106. Other patterns for the micro-needles 112 include, but are not limited to, elliptical, oval or polygonal, where the patterns can be eccentric or centric relative the geometric center of the second major surface 120 of the base 106.

The width of the infiltration area 146 defined by the pattern of the micro-needles 112 can be from 2 mm to 10 mm. So, when the micro-needles 106 are arranged in a circular pattern the infiltration area can be from 3.14 mm² to 78.5 mm². Preferably, the pattern of the micro-needles 106 provides a width (e.g., a diameter) of the infiltration area of 6 mm. Micro-needles 106 can be spaced, on center of their longitudinal axis from each other, in a range from 1 mm to 5 mm. Preferably, the micro-needles 106 can be spaced, on center of their longitudinal axis from each other, in a range from 1.5 mm to 2 mm.

As for the micro-needles 112, they can have a tip 144 spaced from the exterior surface 120 of the base 106, where the tip 144 has a bevel. Examples of such bevels include, but are not limited to, a true short bevel, a short bevel or a standard bevel as are known. The elongate micro-needles 112 also have an outer diameter in a range of 100 micrometer (μm) to 400 μm. The micro-needles 112 also have a length in a range from 500 μm to 1500 μm. The micro-needles 112 of the micro-needle array 102 can all have the same approximate length so that the tip 144 of micro-needles 112 are all approximately on a common plane. Alternatively, micro-needles 112 of the micro-needle array 102 can have different lengths so that the tips 144 of micro-needles 112 are not all approximately on a common plane.

The different components of the micro-needle array 102 can be formed from a polymeric material. For example, the micro-needle array 102 can be made of a polymer selected from the group consisting of aromatic polyester polymers or polycarbonate polymers. Examples of aromatic polyester polymers include liquid crystal polymers (partially crystalline aromatic polyesters based on p-hydroxybenzoic acid and related monomers), such as those sold under the trade designator “Siveras LX” (Toray), “Sumikasuper” (Sumitomo), “Titan” (DuPont), “Vectra” (Celanese), “Xydar” (Solvay Specialty Polymer) or “Zenite” (Celanese). Suitable examples of polycarbonate polymers include those of medical grade that comply with ISO 10993-1 and/or USP Class VI standards.

Examples of suitable polymers for the liquid connection port 104, the sidewall 108 and the top 110 include polymers selected from the group consisting of high density polyethylene, low density polyethylene, polypropylene, polyethylene terephthalate, aromatic polyester polymers (as provided herein), brominated butyl rubber or acrylonitrile-methyl acrylate copolymer. An example of the acrylonitrile-methyl acrylate copolymer includes BAREX®. The different components of the micro-needle array 102 can be formed as separate structures or different combinations of the components can be formed from a single piece of the polymeric material. For example, the base 106 and the micro-needles 122 can be formed as a first piece of the micro-needle array 102 in an injection molding process, where the openings 116 can directly be injection molded or a laser can be used to form (e.g., drill) the openings 116 of the micro-needles 122. Other techniques for forming the openings 116 are possible, including using a water jet or a plasma cutting operation to form the openings 116.

Similarly, the liquid connection port 104, the sidewall 108 and top 110 can be formed as a second piece of the micro-needle array 102 in an injection molding process. The two pieces of the micro-needle array 102 can then be joined together using a variety of techniques. For example, the two pieces of the micro-needle array 102 can be joined using ultrasonic welding. Alternatively, a medical grade chemical adhesive can be used to join the two pieces of the micro-needle array 102. Examples of such medical grade chemical adhesives include, but are not limited to, cyanoacrylates, epoxies, polyurethanes and silicones, as are known.

The two pieces of the micro-needle array 102 can also be joined using a mechanical interaction. For example, the base 106 and the sidewall 108 can include a screw thread that allows the two pieces to be joined. In this embodiment, the base 106 can include an external thread and the sidewall 108 includes an internal thread that allows the two pieces to be joined together by rotating the two pieces along the threads. If desired, ultrasonic welding and/or a medical grade chemical adhesive can also be used.

In an additional embodiment, the micro-needle device 100 can also include a medical grade pressure sensitive adhesive located at least partially across the exterior surface 124 of the top 110. For example, the medical grade pressure sensitive adhesive can be located across the entirety of the exterior surface 124 of the top 110. The medical grade pressure sensitive adhesive can help to retain the micro-needle device 100 on a user's finger. Examples of suitable medical grade pressure sensitive adhesive include, but are not limited to, rubber or Acrylic ester copolymers, zinc oxide rubber adhesives and polyacrylate adhesives The micro-needle device 100 of the present disclosure can also include a catheter 150, as seen in FIG. 1C. The catheter 150 can be formed of medical grade silicon rubber, nylon, polyvinyl chloride (PVC), polyvinylidene chloride (PVDC), polyurethane or polyethylene terephthalate, among other elastomers useful in the medical arts.

The catheter 150 includes a first end 152, at which the lumen of the catheter 150 can receive the liquid to be injected through the micro-needles 112. The catheter 150 can extend from the liquid connection port 104 to the first end 152, where the catheter 150 provides a fluid connection from the first end 152 to the volume 130 and the micro-needles 112 of the micro-needle array 102.

The catheter 150 further includes a second end 154 of the catheter 150, distal from the first end 152. The second end 154 can be positioned relative the liquid connection port 104 to engage the fluid fitting 140 in a fluid tight manner. For example, the second end 154 of the catheter 150 can be slid over the barbs of the fluid fitting 140 to retain the catheter 150 in a fluid tight manner on the micro-needle device 100. Alternatively, the fluid fitting 140 of the liquid connection port 104 and the second end 154 of the catheter 150 can be configured to engage in a fluid tight manner to allow a liquid to flow through the lumen of the catheter 150 through the opening 116 of the micro-needles 112. An example of such a fluid fitting 140 of the liquid connection port 104 and the second end 154 of the catheter 150 can include the female part and the male part of a Luer Taper connector (either a “Luer-Lok” or “Luer-Slip” configuration) as discussed herein.

A syringe 156 can be used to provide a liquid, such as the dental local anesthetic, through the catheter 150, where the syringe 156 releasably couples to the first end 152 of the catheter 150 to provide the fluid connection with the volume 150 of the micro-needle array 102. The syringe 156 can be releasably joined to the first end 152 of the catheter 150 using a fluid fitting such as a Luer Taper connector (either a “Luer-Lok” or “Luer-Slip” configuration) as discussed herein. The syringe 156 can include the dental local anesthetic. Air can be removed from the syringe 156, the catheter 150 and the micro-needle device 100 by positioning the micro-needles 112 at the highest relative point for these structures and driving any air from the assembly using the syringe 156.

Referring now to FIG. 2, there is shown an embodiment of the micro-needle device 200 according to the present disclosure. The micro-needle array 202 includes, as previously discussed, the micro-needle array 202 and the liquid connection port 204. The micro-needle array 202 includes the base 206, the sidewall 208 and the top 210. The base 206 includes two or more of the elongate micro-needle 212 having the interior surface defining the opening through the elongate micro-needle 212. The elongate micro-needle 212 passes across the base 206, and the interior surface of the sidewall 208, the interior surface of the top 210 and the first major surface of the base 206 define a volume, as discussed herein. The liquid connection port 204 includes the lumenal surface 232 defining a lumen 234 that is in fluid connection with the volume of the micro-needle array 202. This allows dental local anesthetic fed through the liquid connection port 204 to pass through the lumen 234, into the volume and exit through the opening of the elongate micro-needle 212. The micro-needle array 202 of the present disclosure has a disk-shape, as previously discussed.

In addition to the structures and advantages discussed for the micro-needle device of the present disclosure, the micro-needle array 202 further includes a spring 260. The spring 260 compresses under pressure applied through a user's finger and against the micro-needle array 202 when the micro-needle device 200 is positioned in a mouth of a patient. As illustrated, the spring 260 is a flat spring having a first leaf 262 and a second leaf 264. The first leaf 262 extends from a first side 266 of the micro-needle array 202 and the second leaf 264 extends from a second side 268 of the micro-needle array 202 opposite of the first leaf 262. Each of the first leaf 262 and the second leaf 264 has an arc-shape that extends from their respective sides in opposite directions. The first leaf 262 and the second leaf 264 arch back over to join a button 269 that is located over the top 210 and the base 206 of the micro-needle array 202. As illustrated, the button 269 is located at a relative low point in the spring 260, which provides both a non-visual guide for the user's finger and allows for greater lateral stability when pressing on the button 269

When the micro-needle device 200 is positioned in a mouth of a patient the user presses on the button 269, which causes the first leaf 262 and the second leaf 264 to bend (the spring 260 compresses). As force is applied to the button 269 the first leaf 262 and the second leaf 264 bend until the button 269 contacts a protrusion 272 on the top 210 of the micro-needle array 202. The protrusion 272 provides the user tactile feedback that the button 269 is in contact with the top 210 of the micro-needle array 202. Contact between the button 269 and protrusion 272 also signals the user that they should not apply any additional pressure to the button 269 as the first leaf 262 and the second leaf 264 have reached the force limit and will not compress any further by applying force to the button 269.

In an alternative embodiment, the button 269 can further include a surface defining an opening through the button 269, where the protrusion 272 can pass at least partially through the opening in the button 269 when the spring 260 is compressed under pressure applied through the button 269 and against the micro-needle array 202 when the micro-needle device 200 is positioned in a mouth of a patient. FIG. 3, and the accompanying discussion, provide a further illustration of this embodiment for the micro-needle device 200.

The amount of force required to bend the first leaf 262 and the second leaf 264 to the point that the button 269 touches the protrusion 272 can be adjusted, as desired, to ensure that the micro-needles 212 of the micro-needle device 200 fully insert into the gingiva and/or other tissues (e.g., oral mucosa) in order to deliver a local anesthetic. This amount of force can also be adjusted to allow the dental professional to better gauge when to stop applying force when using the micro-needle device 200. The height of the protrusion 272 can be designed to set the force threshold for force limitation before the tactile feedback signal is sent. Such adjustments to the required force can be made by changes in any one of the cross-sectional size and/or shape of the first leaf 262 and the second leaf 264. As illustrated, the first leaf 262 and the second leaf 264 each have a rectangular cross-section. It is appreciated that other cross-sectional shapes for the first leaf 262 and the second leaf 264 are possible. Examples include, but are not limited to circular, oval or polygonal, among others.

Additionally, the material from which the first leaf 262 and the second leaf 264 are formed can also be used to adjust the amount of force required to bend the first leaf 262 and the second leaf 264. The shape and size of each of the first leaf 262 and the second leaf 264 can also be used to adjust the amount of force required to bend the first leaf 262 and the second leaf 264. Preferably, the amount of force required for bending the first leaf 262 and the second leaf 264 is from 2 to 20 Newtons.

The first leaf 262, the second leaf 264, the button 269 and the protrusion 272 can each be formed from the same polymeric material during the same process used to form the top 210 of the micro-needle array 202. In an additional embodiment, the button 269 can also include a medical grade pressure sensitive adhesive, as discussed herein, located at least partially across an exterior surface 274 of the button 269. The medical grade pressure sensitive adhesive can help to retain the micro-needle device 200 on a user's finger. Examples of medical grade pressure sensitive adhesives include rubber or Acrylic ester copolymers, zinc oxide rubber adhesives and polyacrylate adhesives.

Referring now to FIG. 3, there is shown an embodiment of the micro-needle device 300 according to the present disclosure. The micro-needle array 302 includes, as previously discussed, the micro-needle array 302 and the liquid connection port 304. The micro-needle array 302 includes the base 306, the sidewall 308 and the top 310. The base 306 includes two or more of the elongate micro-needle 312 having the interior surface defining the opening through the elongate micro-needle 312. The elongate micro-needle 312 passes across the base 306, and the interior surface of the sidewall 308, the interior surface of the top 310 and the first major surface of the base 306 define a volume, as discussed herein. The liquid connection port 304 includes the lumenal surface 332 defining a lumen 334 that is in fluid connection with the volume of the micro-needle array 302. This allows dental local anesthetic fed through the liquid connection port 304 to pass through the lumen 334, into the volume and exit through the opening of the elongate micro-needle 312. The micro-needle array 302 of the present disclosure has a disk-shape, as previously discussed. The micro-needle array 302 further includes the spring 360, as previously discussed.

The micro-needle device 300 further includes a finger ring 374 that extends from the spring 360. The finger ring 374 can, among other things, hold a user's finger against the button 369. As illustrated, the finger ring 374 includes a first arm 376 and a second arm 378 that form a hoop 380 of the finger ring 374. The first arm 376 and the second arm 378 each include an end 382, where the end 382 of each of the first arm 376 and the second arm 378 are free so as to allow the hoop 380 of the finger ring 374 to have an adjustable diameter.

FIG. 3 also illustrates an embodiment in which the button 369 has a surface 384 defining an opening 386 through the button 369. The protrusion 372 can pass at least partially through the opening 386 in the button 369 when the spring 360 is compressed under pressure applied through the button 369 and against the micro-needle array 302 when the micro-needle device 300 is positioned in a mouth of a patient. So, for example, the protrusion 372 can have a diameter and a height that allows it to pass through the opening 386 so the user can first feel the protrusion 372 before the button 369 touches the top 310. Allowing this to happen provides the user tactile feedback that the button 369 is almost in contact with the top 310 of the micro-needle array 302.

In an additional embodiment, the button 369 can also include a medical grade pressure sensitive adhesive, as discussed herein, located at least partially across an exterior surface 374 of the button 369. The medical grade pressure sensitive adhesive can help to retain the micro-needle device 300 on a user's finger.

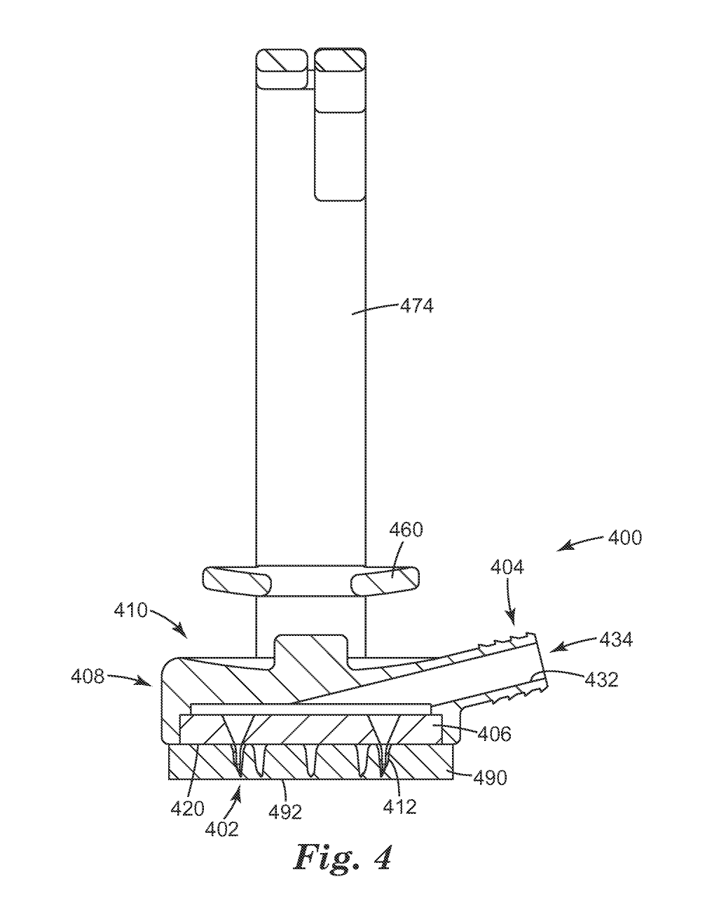

Referring now to FIG. 4, there is shown an additional embodiment of the micro-needle device 400 according to the present disclosure. The micro-needle array 402 includes, as previously discussed, the micro-needle array 402 and the liquid connection port 404. The micro-needle array 402 includes the base 406, the sidewall 408 and the top 410. The base 406 includes two or more of the elongate micro-needle 412 having the interior surface defining the opening through the elongate micro-needle 412. The elongate micro-needle 412 passes across the base 406, and the interior surface of the sidewall 408, the interior surface of the top 410 and the first major surface of the base 406 define a volume, as discussed herein. The liquid connection port 404 includes the lumenal surface 432 defining a lumen 434 that is in fluid connection with the volume of the micro-needle array 402. This allows dental local anesthetic fed through the liquid connection port 404 to pass through the lumen 434, into the volume and exit through the opening of the elongate micro-needle 412. The micro-needle array 402 of the present disclosure has a disk-shape, as previously discussed. The micro-needle array 402 further includes the spring 460 and the finger ring 474, as previously discussed.

As illustrated in FIG. 4, the second major surface 420 of the base 406 can further include a compressible coat 490 that surrounds the micro-needles 412. The compressible coat 490 is formed from a foamed elastic material that is compressible. Examples of such a foamed elastic material include viscoelastic polyurethane foams and low-resilience polyurethane foams. The compressible coat can also be formed from foams of polystyrene, polypropylene, polyethylene or polymers of other vinyl monomers as are known.

The compressible coat 490 has an outer surface 492. As illustrated in FIG. 4, each tip of the micro-needle 412 does not extend above the outer surface 492 of the compressible coat 490. The compressible coat 490 can change shape under a compressive force, allowing the micro-needles 412 to extend beyond the outer surface 492 of the compressible coat 490.

1. A micro-needle device for delivering a dental local anesthetic, comprising:
a micro-needle array having a base, a sidewall and a top, where:
the base includes two or more of an elongate micro-needle, the elongate micro-needle having an interior surface defining an opening through the elongate micro-needle and the base having a first major surface and a second major surface through which the opening of the elongate micro-needle passes to provide a passage across the base;
the top having an interior surface; and
the sidewall having an interior surface, where the interior surface of the side wall, the interior surface of the top and the first major surface of the base define a volume; and
a liquid connection port in fluid connection with the volume of the micro-needle array such that dental local anesthetic fed through the connection port can exit through the opening of the elongate micro-needle.

2. The micro-needle device of claim 1, where the liquid connection port extends from the sidewall of the micro-needle array.

3. The micro-needle device of claim 1, where the micro-needle array further includes a spring that connects the micro-needle array and a button positioned over the top of the micro-needle array, where the spring compresses under pressure applied through the button and against the micro-needle array when the micro-needle device is positioned in a mouth of a patient.

4. The micro-needle device of claim 3, where the top includes an exterior surface opposite the second major surface of the base, the exterior surface of the top having a protrusion that extends towards the button positioned over the top of the micro-needle array.

5. The micro-needle device of claim 3, where the micro-needle array includes a finger ring that extends from the spring, where the finger ring holds a finger against the button.

6. The micro-needle device of claim 5, where the finger ring includes a first arm and a second arm that form a hoop of the finger ring.

7. The micro-needle device of claim 6, where the first arm and the second arm each include an end, where the end of each of the first arm and the second arm are free so as to allow the hoop of the finger ring to have an adjustable diameter.

8. The micro-needle device of of claim 3, where the button has a surface defining an opening through the button, where the protrusion passes at least partially through the opening in the button when the spring is compressed under pressure applied through the button and against the micro-needle array when the micro-needle device is positioned in a mouth of a patient.

9. The micro-needle device of claim 3, where the spring is a flat spring having a first leaf and a second leaf, where the first leaf extends from a first side of the micro-needle array to the finger ring and the second leaf extends from a second side of the micro-needle array opposite of the first leaf.

10. The micro-needle device of claim 1, where the second major surface of the base includes an outer boundary that extends radially from the two or more of the elongate micro-needle to define an infiltration area, and where the top includes an exterior surface opposite the second major surface of the base, the exterior surface of the top providing a continuous surface on which can received pressure from a finger and where the exterior surface opposite of the second major surface and the infiltration area overlap each other by at least 75%.

11. The micro-needle device of claim 1, where the top includes an exterior surface opposite the second major surface of the base, the exterior surface of the top having a pressure sensitive adhesive for retaining the micro-needle device on a user's finger.

12. The micro-needle device of claim 1, where the base, the sidewall and the top of the micro-needle array have a disk-shape.

13. The micro-needle device of claim 12, where the base and the top of the micro-needle array in the disk-shape have a diameter of 4 millimeters (mm) to 15 mm and the sidewall has a height of 0.5 mm to 8 mm.

14. The micro-needle device of claim 12, where the base of the micro-needle array has 6 to 18 micro-needles.

15. The micro-needle device claim 14, where the micro-needles are uniformly arranged in a circular pattern to define the infiltration area.

16. The micro-needle device of claim 1, where the second major surface of the base further includes a compressible coat surrounding the micro-needles, the compressible coat formed from a foamed elastic material and having an outer surface, where the compressible coat changes shape under a compressive force to allow the micro-needles to extend beyond the outer surface of the compressible coat.

17. The micro-needle device of claim 16, where each micro-needle includes a tip that does not extend above the outer surface of the compressible coat.

18. The micro-needle device of claim 1, where the micro-needle device includes a catheter that extends from the liquid connection port to a first end, where the catheter provides a fluid connection from the first end to the volume of the micro-needle array.

19. The micro-needle device of claim 18, further including a syringe that releasably couples to the first end of the catheter to provide the fluid connection with the volume of the micro-needle array.

20. The micro-needle device of claim 19, where the syringe includes the dental local anesthetic.
